(19) United States
(12) Patent Application Publication
(10) Pub. No.: US 2012/0076693 A1
(43) Pub. Date: Mar. 29, 2012

(54) CAPILLARY FLOW TEST ASSEMBLY

(75) Inventor: William R. Hopper, East Ivanhoe (AU)

(73) Assignee: AXXIN PTY LTD, Richmond (AU)

(21) Appl. No.: 13/266,156

(22) PCT Filed: Apr. 27, 2010
(86) PCT No.: PCT/AU2010/000483

§ 371 Date: Dec. 2, 2011

(30) Foreign Application Priority Data
Apr. 27, 2009 (AU) 2009901810

(51) Int. Cl. G01N 33/50

(52) U.S. Cl. 422/68.1

(57) ABSTRACT

A capillary flow test assembly, including: a receptacle having an opening to receive a sample collection device including a sample to be tested using a capillary flow test, the receptacle containing: (i) one or more chemical entities to interact with the sample prior to performing the test, including one or more liquids contained in one or more packages; (ii) a release component configured to release the one or more liquids from the one or more packages to allow the one or more chemical entities to interact with the sample and thereby provide at least one interaction product for the test; (iii) a capillary flow test pad assembly configured to receive the at least one interaction product and to perform the capillary flow test thereon; and (iv) a delay component configured to delay or prevent transport of the one or more chemical entities and the sample to the capillary flow test pad assembly to allow time for the interaction to occur.

### TECHNICAL FIELD

The present invention relates to the general fields of diagnostic and biomedical testing, using immunoassay or capillary flow test strips, and in particular to a capillary flow test assembly, which may be a consumable and compact portable test device suitable for use in medical diagnostics at the Point-of-Care (POC) and in Physician's Office Laboratories (POL) using very low cost components.

### BACKGROUND

As described in the Wikipedia¹ at http://en.wikipedia.org/wiki/Immunoassay:
“An immunoassay test is a biochemical test that measures the concentration of a substance in a biological liquid, typically serum or urine, using the reaction of an antibody or antibodies to its antigen. The assay takes advantage of the specific binding of an antibody to its antigen. Monoclonal antibodies are often used as they only usually bind to one site of a particular molecule, and therefore provide a more specific and accurate test, which is less easily confused by the presence of other molecules. The antibodies picked must have a high affinity for the antigen (if there is antigen available, a very high proportion of it must bind to the antibody). ¹ The Wikipedia text quoted herein is released under CC-BY-SA, see http://creativecommons.org/licenses/by-sa/3.0.
Both the presence of antigen or antibodies can be measured. For instance, when seeking to detect the presence of an infection the concentration of antibody specific to that particular pathogen is measured. For measuring hormones such as insulin, the insulin acts as the antigen.
For numerical results, the response of the fluid being measured must be compared to standards of a known concentration. This is usually done though the plotting of a standard curve on a graph, the position of the curve at response of the unknown is then examined, and so the quantity of the unknown found.
Detecting the quantity of antibody or antigen can be achieved by a variety of methods. One of the most common is to label either the antigen or antibody. The label may consist of an enzyme, enzyme immunoassay (EIA)), radioisotopes such as I-125 Radioimmunoassay (RIA), magnetic labels (magnetic immunoassay—MIA) or fluorescence. Other techniques include agglutination, nephelometry, turbidimetry and Western Blot. A number of these do form a directly visible line or test output but require an instrument to measure or capture the test output.
Immunoassays can be divided into those that involve labelled reagents and those which involve non-labelled reagents. Those which involve labelled reagents are divided into homogenous and heterogeneous (which require an extra step to remove unbound antibody or antigen from the site, usually using a solid phase reagent) immunoassays. Heterogeneous immunoassays can be competitive or non-competitive.
In a competitive immunoassay, the antigen in the unknown sample competes with labelled antigen to bind with antibodies. The amount of labelled antigen bound to the antibody site is then measured. In this method, the response will be inversely proportional to the concentration of antigen in the unknown. This is because the greater the response, the less antigen in the unknown was available to compete with the labelled antigen.
In non-competitive immunoassays, also referred to as the “sandwich assay,” antigen in the unknown is bound to the antibody site, and then labelled antibody is bound to the antigen. The amount of labelled antibody on the site is then measured. Unlike the competitive method, the results of the non-competitive method will be directly proportional to the concentration of the antigen. This is because labelled antibody will not bind if the antigen is not present in the unknown sample.

Because homogeneous assays do not require this step, they are typically faster and easier to perform.”

As described in the Wikipedia¹ at http://en.wikipedia.org/wiki/Lateral flow test:
“Lateral flow tests also known as Lateral Flow Immunochromatographic Assays are a simple device intended to detect the presence (or absence) of a target analyte in sample (matrix). Most commonly these tests are used for medical diagnostics either for home testing, point of care testing, or laboratory use. Often produced in a dipstick format, Lateral flow tests are a form of immunoassay in which the test sample flows along a solid substrate via capillary action. After the sample is applied to the test it encounters a coloured reagent which mixes with the sample and transits the substrate encountering lines or zones which have been pre-treated with an antibody or antigen. Depending upon the analytes present in the sample the coloured reagent can become bound at the test line or zone. Lateral Flow Tests can operate as either competitive or sandwich assays.
In principle any coloured particle can be used, however most tests commonly use either latex (blue colour) or nanometre sized particles of gold (red colour). The gold particles are red in colour due to localized surface Plasmon resonance. Fluorescent or magnetic labelled particles can also be used—however these require the use of an electronic reader to access the test result.
The sample first encounters coloured particles which are labelled with antibodies raised to the target analyte. The test line will also contain antibodies to the same target, although it may bind to a different epitope on the analyte.
The test line will show as a coloured band in positive samples.
The sample first encounters coloured particles which are labelled with the target analyte or an analogue. The test line contains antibodies to the target/its analogue. Unlabelled analyte in the sample will block the binding sites on the antibodies preventing uptake of the coloured particles.
The test line will show as a coloured band in negative samples.
Most tests are intended to operate on a purely qualitative basis. However it is possible to measure the intensity of the test line to determine the quantity of analyte in the sample. Implementing a Magnetic immunoassay (MIA) in the lateral flow test form also allows for getting a quantified result.
While not strictly necessary, most tests will incorporate a second line which contains an antibody that picks up free latex/gold in order to confirm the test has operated correctly . . .
Time to obtain the test result is a key driver for these products. Tests can take as little as a few minutes to develop. Generally there is a trade-off between time and sensitivity—so more sensitive tests may take longer to develop. The other key advantage of this format of test compared to other immunoassays is the simplicity of the test—typically requiring little or no sample or reagent preparation . . .
Probably the most well known examples of lateral flow tests are home pregnancy tests. However rapid tests or point of care tests are available for a wide range of applications including: HIV tests, Troponin T, test Malaria tests, drugs of Abuse tests, Fertility tests, Respiratory disease tests etc. Clinical tests can be applied to urine, saliva, blood, or stool samples. Tests are available for both human and animal diagnostics. Tests are also available for non clinical applications including testing food and water for contaminants.”

FIG. 1 shows a typical prior art lateral or capillary flow pad assembly or strip as commonly used in rapid diagnostic applications. The strip contains an absorptive sample application or input pad 102, a conjugate pad 104, a membrane 106 along which the analyte flows, and a waste adsorbing pad 108. These components are bonded by an adhesive layer 110, onto a carrier strip 112, usually constructed from plastic sheet.

Immobilised on the membrane (typically nitrocellulose) are one or several test regions or line(s) 114 containing capture antigens or antibodies for the target(s) of interest, and a control region or line 116 containing a control capture antigen or antibody. As described above, visible or colored or fluorescent labels are incorporated, such that the test result is displayed as one or more visible or otherwise optically detectible lines at the test region(s) 114 and/or the control region 116.

Lateral flow strips such as that shown in FIG. 1 are often contained in a plastic cassette having an opening for sample introduction and a open or transparent “window” for viewing the test and control lines 114, 116.

Currently, lateral flow and other similar types of biomedical test strips are widely used to diagnose a range of medical conditions from pregnancy, health markers and infectious diseases, for example flu. Although some tests do not require sample preparation, in many cases, the sample must be washed and treated by reagents before being applied to the lateral flow test strip in order to enable the required operation, sensitivity or reliability of the test. This requirement to prepare a treated or processed liquid suspended preparation of the original sample can render such tests challenging to inexpert users and risks the test results being inaccurate or misleading due to incorrect sample preparation on the part of the user. For example, in the case of a test that uses a nasal or mouth swab sample, the sample must be washed from the swab and stabilized in a liquid suspension in order to be in a form that is suitable for capillary flow through a test strip.

For this reason, a typical test kit for such tests includes a small box containing separate apparatus and reagents that a user must individually open and use. Typically, such a test kit will contain a test tube or molded consumable and separate powder and liquid reagents, along with a separate package that contains the test strip. The reagents are thus supplied separately from the test tube and must be added to the tube by the user. Only then can the user insert the swab to wash and treat its captured sample prior to manually introducing the absorption pad 102 of the lateral flow strip to the prepared sample liquid, to commence the capillary flow required for the test strip to determine a result. Moreover, a specified time delay is typically required from the time the sample is washed in the reagents to the time the strip is introduced to allow adequate time for mixing of the sample and reagents, and reaction of these components. The user must therefore manually manage the mixing and timing of this relatively complex sample preparation stage, making the test difficult to perform correctly and hence prone to user error.

It is desired to provide a capillary flow test assembly that alleviates one or more of the above difficulties, or at least provides a useful alternative.

### SUMMARY

In accordance with the present invention, there is provided a capillary flow test assembly, including:
a receptacle having an opening to receive a sample collection device including a sample to be tested using a capillary flow test, the receptacle containing:
   one or more chemical entities to interact with the sample prior to performing the test, including one or more liquids contained in one or more packages;
   a release component configured to release the one or more liquids from the one or more packages to allow the one or more chemical entities to interact with the sample and thereby provide at least one interaction product for the test;
   a capillary flow test pad assembly configured to receive the at least one interaction product and to perform the capillary flow test thereon; and
   a delay component configured to delay or prevent transport of the one or more chemical entities and the sample to the capillary flow test pad assembly to allow time for the interaction to occur.

Embodiments of the present invention include low cost, disposable consumable assemblies that allow all of the sample washing, reagent addition, mixing and time delays to be automatically managed in a single step, easy to use, point of care or diagnostic capillary flow type test. The consumables can be used to provide ease of use for a manually read test format, and/or used in combination with a reader test instrument that incorporates features to mix, monitor, detect and optimally read the diagnostic or biological test without user intervention following addition of the test sample.

Also described herein is a capillary flow test assembly, including:
a receptacle having an opening to receive a sample collection device including a sample to be tested using a capillary flow test, the receptacle containing:
one or more chemical entities to interact with the sample prior to performing the test, including one or more chemical entities contained in one or more packages;
a release component configured to release the one or more chemical entities from the one or more packages to allow the one or more chemical entities to interact with the sample and thereby provide at least one interaction product for the test;
a capillary flow test pad assembly configured to receive the at least one interaction product and to perform the capillary flow test thereon; and
a delay component configured to delay or prevent transport of the one or more chemical entities and the sample to the capillary flow test pad assembly to allow time for the interaction to occur.

Also described herein is a capillary flow test assembly, including:
a receptacle having an opening to receive a sample collection device including a sample to be tested using a capillary flow test, the receptacle. containing:
one or more chemical entities to interact with the sample to provide at least one interaction product for the test;
a capillary flow test pad assembly configured to receive the at least one interaction product and to perform the capillary flow test thereon; and
a delay component configured to delay or prevent transport of the one or more chemical entities and the sample to the capillary flow test pad assembly to allow time for the interaction to occur.

One or more of the one or more chemical entities may be provided in one or more packages within the assembly, and the assembly may include a release component configured to release the one or more chemical entities from the one or more packages to allow the one or more chemical entities to interact with the sample and thereby provide at least one interaction product for the test.

The one or more chemical entities contained in the one or more packages may be in liquid, powder, or other solid form.

### BRIEF DESCRIPTION OF THE DRAWINGS

Some embodiments of the present invention are hereinafter described, by way of example only, with reference to the accompanying drawings, wherein:

FIG. 1 is a schematic diagram of a prior art lateral flow test strip device;

FIG. 2 is an image of an embodiment of a capillary flow test assembly or device;

FIG. 3 is an image of a swab assembly of the device;

FIG. 4 is an image of the device with the swab assembly coupled thereto;

FIG. 5 is an image showing (from left to right) a capillary flow test strip, an insert, and a reagent package or sachet of the device;

FIG. 6 is an image showing a cross-sectional view various internal components of the device, including perforation of the reagent sachet within the device;

FIGS. 7 and 8 are images showing transverse and longitudinal cross-sectional views, respectively, of an alternative embodiment of a capillary flow test device or assembly;

FIG. 9 is an image of a further alternative embodiment of a capillary flow test device or assembly;

FIG. 10 is an image of the device of FIG. 9 with the swab fully inserted therein;

FIG. 11 is an image of an embodiment of a capillary flow test device or assembly having a machine-readable barcode;

FIG. 12 is an image of an embodiment of a capillary flow test reader desktop instrument for use with the capillary flow test devices described herein; and

FIG. 13 is an image of a removable carrier of the instrument of FIG. 12.

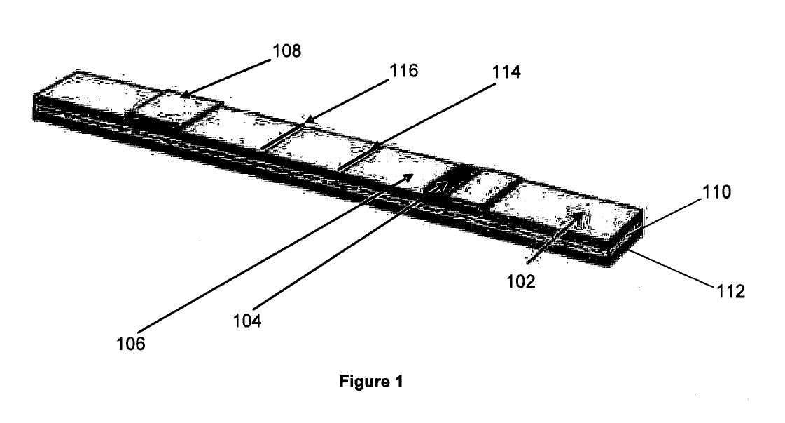

### DETAILED DESCRIPTION

Embodiments of the present invention include assemblies, devices, and systems for performing lateral or capillary flow tests requiring sample preparation in an automated or semi-automated manner. In particular, these assemblies, devices, and systems include all of the reagents (or other chemical entities) required for sample preparation and the capillary flow test pad assembly (usually but not necessarily in the form of an elongate strip referred to in the art as a ‘lateral flow test strip’, notwithstanding that the orientation of the strip, and hence the flow, may be vertical), supplied to a user in a single receptacle, container and/or cartridge, where one or more liquid reagents are packaged and protected for storage and the test pad assembly is sealed and protected from the environment for storage and transport.

At the time that a test is required, the user can simply insert a sample collection device including a biological sample such as a nasal swab into the device or system, in response to which all of the fluid and/or chemical dispensing, sample washing, mixing, time delays, and finally the introduction of the resulting test fluid to the test strip are automatically undertaken by the components within the device or system.

In general, capillary flow test assemblies or devices in accordance with various embodiments of the present invention include a receptacle having an opening to receive a sample collection device in or on which a sample to be tested has been collected for testing. The receptacle contains one or more chemical entities to interact with the sample prior to performing a capillary flow test, including one or more chemical entities contained in one or more packages or sachets. Typically, each package includes a chemical entity or entities in liquid form, although a package may include an entity or entities in powder or other solid form. In some tests, the sample itself is in liquid form and provides liquid for mixing. The chemical entities typically include one or more reagents, but in some embodiments only non-reagent chemical entities may be provided. The one or more liquids typically include one or more of the reagents, but in some cases may be water or a solvent or some other non-reagent liquid such as a buffer or diluent that releases or otherwise activates, dissolves, and/or mixes with one or more of the reagents or other chemical entities provided in solid or powder form. Also included within the receptacle is a release component configured to release the one or more liquids from the one or more sachets to allow the chemical entities to react or otherwise interact with the sample and thereby provide at least one interaction product for the test. The at least one interaction product can include a reaction product or can be a filtered component of the original sample, for example. The interaction product is subsequently provided to a capillary flow test pad assembly or strip to perform the desired capillary flow test. However, in order to ensure that the sample and reagents have had adequate time to interact, the device also includes a delay component configured to delay or prevent transport of the reagents and the sample to the capillary flow test pad assembly.

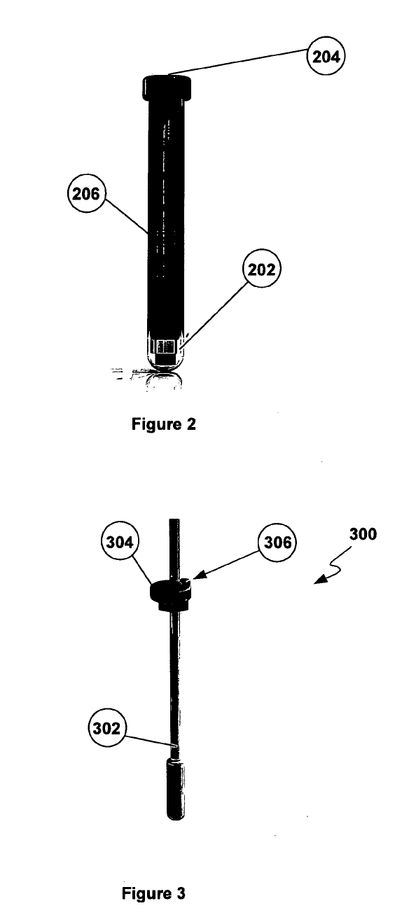

As shown in FIG. 2, in some embodiments the receptacle is provided in the form of a glass or plastic tube 202, which can be a standard glass test tube, containing sample preparation and capillary flow test components. In some embodiments, the tube 202 is a standard glass test tube of 10 mm diameter and length 75 mm. The device comes sealed by an elastomeric or plastic cap 204 and contains a capillary flow test strip 206, one or more reagents for use in preparing the sample for the capillary flow test, and other sample preparation components, as described below.

In some embodiments, the device includes a sample collection device 302 for collecting a sample for testing. In some embodiments, the sample collection device is in the general form of a swab 302 with an elongate handle or shaft, and may include a plastic sealing cap 304 having an opening through which the shaft of the swab 302 passes, as shown in FIG. 3, with the plastic cap 304 forming a seal with the shaft. In some embodiments, the sample collection device or swab 302 and sealing cap 304 are provided together as a sample collection assembly 300. In some embodiments, the shaft of the swab 302 has a larger diameter at the end opposite to the sample collecting end or a retaining collar or similar feature disposed on the shaft so that the swab 302 is permanently attached to the sealing cap 304. In other embodiments, the swab 302 can be readily slid from the opening in the sealing cap 304. In some embodiments, the sealing can 304 has an opening that readily accommodates and forms a seal with a standard commercially available swab.

More generally, the sample collection device is not restricted to the form of a swab as generally shown in the Figures, but may take other forms, including forms incorporating sample collection features such as internal cavities, absorption pads and/or other features to collect any environmental or biological fluid sample, including blood or urine, for example.

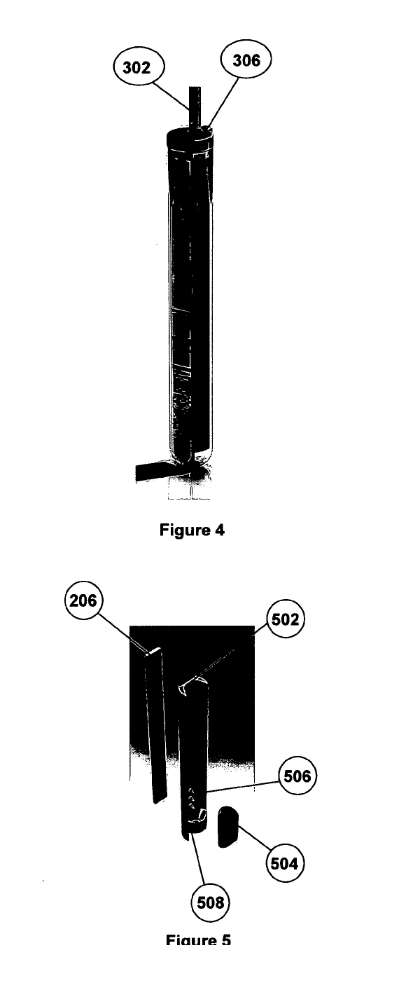

Returning to the embodiment shown in FIG. 3, once the swab 302 has been used to collect a sample (e.g., a nasal sample), the swab 302 is inserted into the tube 202 and its cap 304 seated on the opening of the tube 202 to form a seal therewith, as shown in FIG. 4. In some embodiments, the cap 304 and/or tube incorporates at least one tab or alignment feature 306 that ensures that when the device is inserted into a handheld or desktop instrument or reader, as described below, the test strip 206 will be correctly positioned for sensors and/or imaging or mixing components incorporated within the instrument.

As shown in FIG. 5, in some embodiments an elongate moulded plastic insert 502 within the tube 202 effectively divides the internal volume of the tube 202 into two portions or cavities, one portion or cavity being used for sample preparation and the other containing the capillary flow test pad assembly or strip 206. The insert 502 forms a fluid tight seal with the internal wall of the tube 202 that prevents the sample and reagents in the sample preparation cavity from passing into the capillary flow test cavity, except for a passage defined at the bottom of the tube 202, as described below. The insert 502 also secures the capillary flow test strip 206 in the correct position by engaging it between the insert 502 and the inner wall of the tube 202.

The device includes one or more reagents or other chemical species that react or otherwise interact with the sample to prepare it or to derive from it a prepared sample suitable for testing by the capillary flow test assembly. The device includes one or more liquids pre-packaged in one or more sealed plastic or foil packages or sachets 504 within the sample preparation cavity. As described above, the liquids typically include at least one of the one or more reagents, although it is possible that the one or more liquids can be water and/or some other form of non-reagent liquid that releases and/or activates one or more of the reagents in powder or other solid form. In some embodiments, the sachets 504 are positioned on or attached to the insert 502 by mechanical engagement, adhesive attachment, or by plastic welding of an edge of each sachet to the insert 502.

As described above, the device includes a release component to release the liquid reagent(s) from the package(s) to allow them to react or otherwise interact with the sample. In some embodiments, the release component is an integral part of the insert 502. In some embodiments, the release component is in the form of one or more projections or pins 506, such as those shown in FIG. 5. When the sample collection device (e.g., the sample swab 302) is inserted into the sample preparation cavity, the package or sachet 504 is forced against the projections 506 and is thereby perforated or ruptured, releasing the contents of the sachet(s) 504.

In some embodiments, the device includes a fluid processing or interaction or sample preparation component to temporary store the reagents and sample so they can interact with each other before being provided to the capillary flow test strip. In some embodiments, this component is in the form or a well located near but spaced from the base of the tube 202 and forming a seal with the inner wall of the tube 302. In the embodiment of FIG. 5, the well 508 is formed as an integral part of the insert 502 for ease of manufacture, although it will be apparent that this need not be the case. In any case, the well 508 is configured so that liquid reagent released from the sachet(s) 504 collects in the well 508 to form a volume of fluid in which the introduced sample or swab is immersed to allow the reagents to interact with the sample.

As described above, it is generally a requirement of such tests that that the reagent(s) and sample are allowed a specified period of time to interact before being provided to the capillary flow test pad assembly 206, and the devices described herein include a delay component that delays or prevents transport of the reagents and the sample to the capillary flow test pad assembly 206.

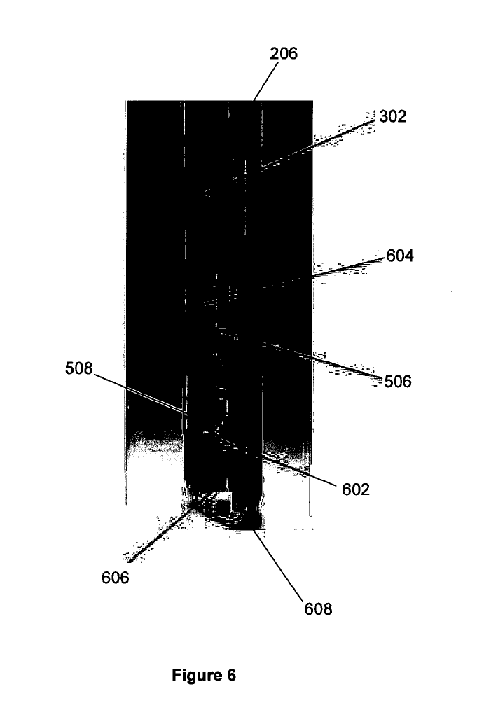

In some embodiments, the delay component is provided in the form of a soluble insert or plug that fills and seals an opening 602 in the base of the fluid containment well 508, as shown in FIG. 6 (plug not shown). This arrangement prevents or inhibits transport of the fluid in the well 508 until a substantial portion of the plug 508 has dissolved, thereby providing the desired time delay, as determined by the composition and configuration of the plug. In the described embodiments, the plug is composed of a soluble material (e.g., sugar) that does not substantially influence the chemistry of the sample preparation. However, in other embodiments, the composition of the plug can provide a chemical component that plays a role in the sample processing.

In any case, the soluble plug delays release of the fluid in the well 508 so that adequate time is provided for: (i) the sample to be washed from the swab 302, (ii) the sample to be adequately mixed with the reagent fluids, and (iii) the reaction(s) to be sufficiently complete prior to absorption and transport along the membranes of the capillary flow test strip 206.

In other embodiments, the delay component can take other forms, including, for example, a fluidic restriction, a fluidic labyrinth or other form of fluidic or microfluidic delay line, or a component such as a permeable membrane or filter (which may be made from a sintered plastic or a metallic material) that allows fluid to pass but imposes a suitable time delay on its passage through the component to provide adequate capture and mixing of the sample with the reagent or wetting liquid.

In some embodiments, the well 508 also includes a mixing component to mechanically mix the reagents and sample. In some embodiments, the mixing component includes a paramagnetic component or bead or magnetic particles that can be excited by a magnetic to assist mixing of the sample and reagents. An externally applied oscillating or AC magnetic field couples with the magnetic particles, causing them to oscillate or vibrate, and thereby assist mixing. In other embodiments, one or more magnetic or paramagnetic particles or components 602 are incorporated into the head of the swab 302, such as the magnetic component 604 shown in FIG. 6, to allow the swab 302 to be vibrated by an externally applied magnetic field to assist washing of the sample from the sample and dispersion into the fluid reagents.

Alternatively, the mixing of reagents and sample can be effected or assisted by vibrating or oscillating the entire tube 202. In various of these embodiments, the mechanical or magnetic oscillations can be provided by a handheld or desktop instrument mechanically coupled to the tube 202 and that can also assist with or provide automatic processing of the sample, or reading of the test result(s).

After a delay period caused by the delay component (corresponding, in some embodiments, to the time taken to dissolve the soluble plug or a substantial portion thereof), the reacted or otherwise interacted reagents and sample produce a fluid including at least one interaction product for the capillary flow test, and that can be considered to represent the prepared sample.

In some embodiments, once a substantial portion of the soluble plug has dissolved, the fluid remaining in the well 508 starts to flow through the hole 602 in the base of the well 508 into the very bottom 606 of the tube 202, thereby flowing into the test portion or test cavity of the tube 202 and onto an absorptive input region 608 of the capillary flow test strip 206. The fluid flows up the test strip 206 under capillary flow in the usual manner so that a biomedical or medical diagnostic test can be performed on the capillary flow strip.

The test result(s) can be read manually or automatically by a reader instrument. The advantage of using an automated instrument to read the test strip is that the test may require time to fully develop, e.g., 10 minutes or more. The reader can be configured to automatically wait the required time for optimal reading of the test strip. Alternatively, a reader instrument can use other strategies such as continually or successively reading the test strip until an optimal contrast is developed, and/or other quantitative or qualitative data can be derived from successive readings.

Another advantage of using an electronic instrument to read the test strip is that it can employ other technologies that do not provide a human readable output, such as fluorescence or magnetic beads or electro chemistry other non visible markers that can be interpreted or detected by the instrument.

Because the sample and reagents are sealed within the device during the test, the entire device can be provided as a consumable item and disposed of as bio-waste following completion of the test, with extremely low risk of spillage or contamination.

Further embodiments of the invention incorporate additional features and/or variations of the features described above to enhance the ease of use and flexibility in some clinical environments.

For example, in some embodiments, the receptacle is a moulded clear plastic tube to avoid the risks associated with glass breakage. In some embodiments, the tube is non-circular in transverse cross-section to assist the internal functions of the device. For example, FIG. 7 shows an embodiment in which the transverse cross-sectional shape of the tube 702 is part-circular and part-rectangular. Tubes with this and similar shapes also assist alignment of the reading areas of the test strip with a reader instrument.

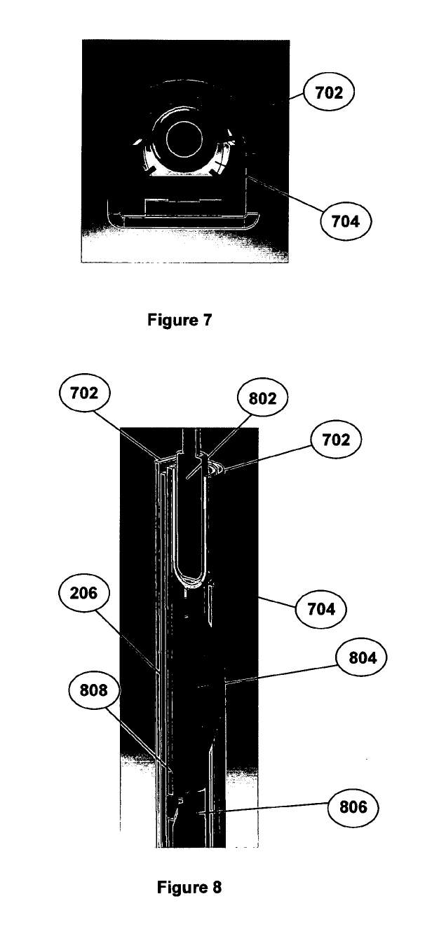

In some embodiments, such as the embodiment shown in FIGS. 7 to 10, the device includes a small moulded plastic perforated or slotted bucket 704 that weakly engaged by a friction fit within the tube 702 and initially secured by friction at or near the top of the tube 702. The bucket 704 is configured to receive a sample collection device or swab 802 and to enclose the swab 802 and travel down the tube 702 with the swab 802 as the swab 802 is inserted into the tube 702. This arrangement has the advantage that the swab 802 does not lose any sample material on the inner wall(s) of the tube 702 or on the reagent sachet(s) as the swab 802 is inserted into the tube 702.

This arrangement also ensures that a fixed and known size (defined by the bucket 704) to the mechanical interaction required to rupture or cut the reagent sachet(s) 804. Provided that the end of the swab 802 or other form of sample collection device tip fits into the bucket 704 and can be used to press the bucket 704 down to the bottom or stop near the bottom of the tube 702, the arrangement will operate regardless of the actual size of the tip of the swab inserted into the tube 702. This arrangement thus allows any swab to be used with the device and can suit typical clinical settings where swabs with different tip size and styles may be used for different collection applications or by specific preferences of clinicians.

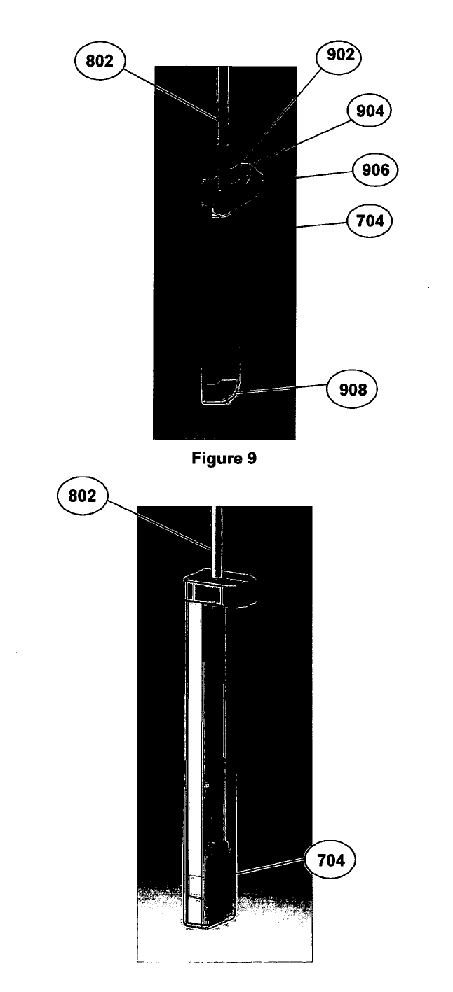

In some embodiments, the device is supplied with a flexible or elastomeric cap 902 molded from a material such as silicone rubber and configured such that it will form a seal with no swab inserted as intended for transport and storage. As shown in FIG. 9, the cap 902 includes a first portion that seals the capillary flow test cavity from the environment, and a second portion operable (by way of a hinge with the first portion) to repeatedly and reversibly open and seal the sample preparation cavity. The second portion includes two or more resiliently deformable sections 904, 906 that form a seal therebetween in the absence of a swab 802, but which conform around and seal against swab shafts of a range of diameters inserted between the sections 904, 906.

As the swab 802 is used to press the bucket 704 to the bottom of the tube 702, it either transports or encounters at least one reagent sachet 804 on its passage down the tube 702. As with some of the embodiments described above, the device includes a projection or spike 806 projecting into the sample preparation cavity and that is configured to cut, rip or otherwise rupture the sachet 804 on its way down the tube 702, thereby releasing the sachet's contents. However, the projection 806 is deformable or otherwise movable (e.g., by being mounted at the end of a resilient arm) such that, although being configured to rupture the sachet, further application of pressure to the end of the swab 802 moves the projection 806 out of the way of the bucket 704, thereby allowing the bucket 704 to pass further into the tube 702.

On reaching the bottom of the tube 702 (or, in some embodiments, on reaching a stop spaced from the bottom of the tube 702), the released liquid reagents fill a bottom portion of the tube 702 and flow into the bucket 704 through its slots or perforations to allow washing and mixing of the sample carried on the swab 802. In some embodiments, this mixing action is mechanically and/or magnetically assisted as described above.

As with some of the embodiments described above, the device includes a delay component to delay transport of the reagents and the sample to the capillary flow test strip 206. In some embodiments, the delay component is in the form of a soluble plug 908 that prevents direct transmission of the fluid onto the absorptive input portion 102 of the test strip 206 until a suitable delay period has elapsed.

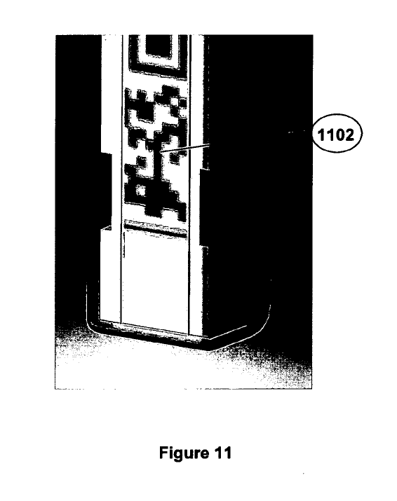

FIG. 10 is an image of the device after the bucket 704 has been pressed down to the bottom of the tube 702 by insertion of the sample swab 802 such that the reagent sachet 804 has been ruptured and the reagent mix is contained in the bottom of the tube 702 and in contact with the sample and a soluble plug 908 or other form of fluid delay that in time allows access of the mix to an absorption portion at the base of the test strip 206.

The capillary flow test devices or assemblies described herein allow the reagents to be assembled as sub-assemblies prior to assembly into completed consumables in a factory environment. For example, each package or sachet can be attached by adhesive or welding to the bucket 704. The bucket 704 can be colour coded to indicate a corresponding reagent or test type. Additionally, the soluble plug 908 and the test strip 206 can be pre-assembled into the base of a plastic insert 808 that engages and seals the test strip 206 into the tube 702.

Where additional reagents are required in solid or powder form, these can be provided sealed in the bottom of the tube 702 or captured as one or more pellets or provided as coatings on the inner surface of the bucket 704 itself. As described above, it is not necessary that any of the reagents themselves are provided in liquid form, since the liquid(s) released from the package(s) could be water, one or more solvents, and/or some other form of non-reagent liquid that releases, activates, or mixes the solid reagents so that they can interact with the sample. Moreover, in some embodiments, the capillary flow test assembly or device need not include any reagents, but rather other chemical species that interact with, but do not necessarily react with, the sample to prepare it for testing. For example, the device may include one or more chemical species or entities that form complexes with or trap via Van der Waals forces one or more components of the sample, thus filtering those components so that the remaining component(s) of the sample can be provided to the test stick.

In some embodiments, the receptacle provides an optical path such that a portion of the bucket 704 can be imaged or otherwise sensed by a reader instrument with the test strip 206 facing an imaging component of the reader instrument. The optical path can be provided by an optically transparent window in the insert 808 that engages the test strip 206, or by a tube configured such that diffraction and/or reflection within the tube provides simultaneous viewing of both the edge of the bucket and the test strip 206.

Using this approach, sensing or image analysis can be used to automatically confirm the bucket colour, (and hence reagent and/or test type) installed, and also that the bucket 704 has been correctly and fully inserted into the device. Alternatively, observation by the detection or optical system of the reader that the bucket 704 has been pressed down can be used as a trigger to start a timer in electronics or software to automatically control the recording of the result(s) of the test without user intervention.

In some embodiments, the test strip and/or the receptacle/tube and/or the insert includes machine-readable indicia such as a one- or two- dimensional barcode, so that the same optical detector used to read the test strip can also read details of the test, such as test type, use by date, batch no, unique test serial number, and so on.

In some embodiments, a reagent package or sachet is ruptured by or with the assistance of an external influence from the reader instrument. In some embodiments, this is provided in the form of magnetic manipulation of a magnetically coupled component captured within the sachet such as a sharpened magnetic or paramagnetic insert. Imposition of a magnetic field such as by energizing an electromagnet in the reader generates a force on the sharpened pellet (which may be in the form of a tetrahedral insert), causing it to rupture the sachet. Mechanical oscillation of the insert, such as by application of an alternating magnetic field, assists rupture of the sachet. Alternatively, a metallic insert such as metallic thread or strip within a section of the sachet wall can be heated by induction and cause the sachet wall to melt and rupture.

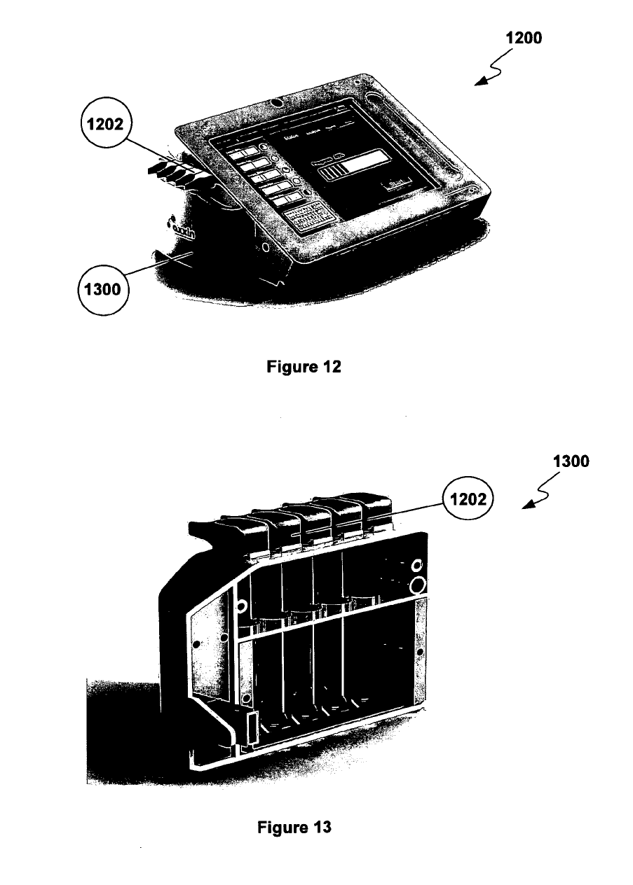

FIG. 12 is an image of an embodiment of a desktop reader instrument 1200 that can simultaneously or sequentially process up to five of the capillary flow test devices or assemblies 1202 described herein retained in a removable carrier 1300 of the instrument 1200, as shown in FIG. 13.

The combination of the reader 1200 and the capillary flow test assemblies or devices 1202 described herein allows a user to simply insert a swab containing a sample into a capillary flow test device 1202 to activate the device 1202, and place the device 1202 into a reader instrument such as the desktop reader 1200 where it will be read when optimally required to be read, thereby adding significantly to the ease of use of such tests.

Many modifications will be apparent to those skilled in the art without departing from the scope of the present invention.

1. A capillary flow test assembly, including:
a receptacle having an opening to receive a sample collection device including a sample to be tested using a capillary flow test, the receptacle containing:
one or more chemical entities to interact with the sample prior to performing the test, including one or more liquids contained in one or more packages;
a release component configured to release the one or more liquids from the one or more packages to allow the one or more chemical entities to interact with the sample and thereby provide at least one interaction product for the test;
a capillary flow test pad assembly configured to receive the at least one interaction product and to perform the capillary flow test thereon; and
a delay component configured to delay or prevent transport of the one or more chemical entities and the sample to the capillary flow test pad assembly to allow time for the interaction to occur.

2. The assembly of claim 1, including a sample processing component configured to receive and contain the sample for interaction with the one or more chemical entities therein, the delay component being configured to delay or prevent transport of the one or more chemical entities and the sample from the sample processing component.

3. The assembly of claim 2, wherein the sample processing component includes a well that forms a seal with an inner wall of the receptacle.

4. The assembly of claim 1, wherein the release component is configured to release the one or more liquids from the one or more packages in response to insertion of the sample collection device into the receptacle.

5. The assembly of claim 1, wherein the release component includes one or more projections configured to rupture the one or more packages.

6. The assembly of claim 5, wherein the one or more projections are initially disposed in a path through the receptacle and are configured to rupture the one or more packages and deform or move out of the path to allow further passage of the sample collection device into the receptacle.

7. The assembly of claim 1, wherein the delay component includes a soluble component that blocks passage of the one or more chemical entities and the sample for a period of time until at least a portion of the soluble component dissolves to allow said passage.

8. The assembly of claim 7, wherein the soluble component is a plug that seals an opening in a sample processing component of the assembly configured to receive the sample for interaction with the one or more chemical entities therein, the delay component being configured to delay transport of the one or more chemical entities and the sample from the sample processing component.

9. The assembly of claim 1, wherein the release component includes a magnetic component configured to effect or facilitate rupture of the packages under the influence of an externally applied magnetic field.

10. The assembly of claim 1, wherein the release component includes a component configured to effect or facilitate rupture of the packages by heating the packages.

11. The assembly of claim 1, including a sample receiving component disposed within the receptacle and configured to receive at least a portion of the sample collection device including the sample therein, said sample receiving component being configured to move from a sample receiving position near the opening of the receptacle to a second preparation position towards a base of the receptacle in response to insertion of the sample collection device into the receptacle; wherein said insertion causes the release of the one or more liquids from the one or more packages such that the one or more chemical entities and the sample interact within the sample receiving component.

12. The assembly of claim 11, wherein the sample receiving component includes one or more openings that allow the released liquids to enter the sample receiving component.

13. The assembly of claim 11, wherein the sample receiving component includes one or more of said chemical entities in solid form.

14. The assembly of claim 11, wherein the sample receiving component includes one or more of said chemical entities in the form of a coating on the sample receiving component.

15. The assembly of claim 11, wherein the sample receiving component includes one or more of said chemical entities in the form of one or more pellets in the sample receiving component.

16. The assembly of claim 1, including a cap that seals the opening of the receptacle, the cap including an opening to seal against a shaft of the sample collection device when disposed within the receptacle.

17. The assembly of claim 16, wherein the cap is configured to receive and seal against shafts of sample collection devices having a range of different diameters.

18. The assembly of claim 16, wherein the cap includes a first portion that seals a test cavity of the receptacle containing the capillary flow test pad assembly, and a second portion that seals a sample preparation cavity of the receptacle containing the one or more chemical entities.

19. The assembly of claim 1, including an insert within the receptacle that engages with an inner wall of the receptacle to define a test cavity of the receptacle containing the capillary flow test pad assembly, and a sample preparation cavity of the receptacle containing the one or more chemical entities, the insert forming a liquid seal between the cavities.

20. The assembly of claim 19, wherein the release component is an integral portion of the insert.

21. The assembly of claim 19, wherein the one or more packages are attached to the insert.

22. The assembly of claim 1, wherein the one or more chemical entities include one or more reagents.

23. The assembly of claim 22, wherein the one or more liquids include at least one of the reagents.
